(19) United States
(12) Patent Application Publication
(10) Pub. No.: US 2023/0173263 A1
(43) Pub. Date: Jun. 8, 2023

(54) SYSTEMS AND METHODS FOR ORAL IONTOPHORESIS

(71) Applicant: BIOLECTRICS LLC, CLEVELAND, OH (US)

(72) Inventors: DAVID J. CORN, CLEVELAND, OH (US); STEVEN W. CORNELIUS, ROCKY RIVER, OH (US); WILLIAM J. LEIMKUEHLER, Jr., AVON, OH (US); ISSAM NEMEH, WESTLAKE, OH (US)

(21) Appl. No.: 17/918,614

(22) PCT Filed: Apr. 16, 2021
(86) PCT No.: PCT/US21/27699

§ 371 Date: Oct. 13, 2022

Related U.S. Application Data
(60) Provisional application No. 63/012,061, filed on Apr. 17, 2020.

(51) Int. Cl. A61N 1/30; A61N 1/05

(52) U.S. Cl. CPC A61N 1/306; A61N 1/0548

(57) ABSTRACT

A method for delivery of oral iontophoretic or reverse-iontophoretic effect by electrical current in combination with electrokinetic elements (e.g., ions, charged molecule(s) (such as a medication or a bioactive agent), and/or charged molecular complexes that may include uncharged molecules), the electrical current causing a motive force on such elements to and from biological tissues and fluids.

### RELATED APPLICATIONS

This application claims the benefit of co-pending international PCT Application No. PCT/US2021/027699, filed Apr. 16, 2021, and entitled “Systems and Methods for Oral Iontophoresis,” which claims the benefit of U.S. Provisional Patent Application Ser. No. 63/012,061, filed Apr. 17, 2020, and entitled “Systems and Methods for Oral Iontophoresis,” both of which are incorporated by reference herein in their entirety.

### BACKGROUND

This invention relates generally to iontophoresis, and more particularly to oral iontophoresis and reverse-iontophoresis, which are methods of electromotive ion transport that involves the application of an electric field for use as the driving forces for transporting ions, charged molecule(s) (such as a medication or a bioactive agent), and/or charged molecular complexes that may include uncharged molecules to and from biological tissues and fluids. Iontophoresis and reverse iontophoresis are nearly identical processes with the same purposes, except that iontophoresis refers to the transport of charged molecules to a biological tissue or fluid, while reverse iontophoresis refers to the transport of charged molecules away from or out of a biological tissue or fluid.

In general an electric field is applied by at least one electrode, preferably at least one pair of electrodes with one such electrode positively charged and the other negatively charged between which lies the aforementioned charged molecule(s), which may be some medium which could be a solution or gel or compound containing charged molecule(s) such as ions, known as electrolytes, with delivery targeted to one or more biological hard and/or soft tissue(s); according to the electrical field applied between the two electrodes, the charged molecule(s) will move/migrate within and through the medium in a process called electrophoresis. In iontophoresis, the electrode that is responsible for propelling the charged molecule toward a biological tissue or fluid is known as the “active” electrode, while in reverse iontophoresis the active electrode is the one responsible for drawing the charged molecule away from the biological tissue or fluid.

Most biological tissues and fluids contain negatively and positively charged molecules such that when subjected to DC stimulation, will cause the migration of these charged particles toward the pole of opposite polarity. Such application may be used to deliberately draw charged molecules out of tissue and fluids or to drive charged molecules into tissues and fluids; in addition to the endogenous charged molecules that can be influenced by DC stimulation, exogenous charge molecules, such as pharmaceutic compounds, can be introduced such that they can be driven into tissues and fluids to achieve therapeutic goals.

The quantity of ions transferred through iontophoresis/reverse-iontophoresis may be determined and/or influenced by the intensity of the applied current, the current density at the active electrode, the duration of stimulation, and the concentration of charged molecules in solution; in general, the number of ions transported is directly proportional to the current density and the duration of stimulation.

### SUMMARY OF THE INVENTION

The present invention relates to a method and apparatus for aiding overall oral health, and more particularly to treating periodontal diseases such as gingivitis, periodontitis, and peri-implantitis; killing oral microbes including cavity-causing bacteria; reducing oral biofilms; increasing blood flow in oral tissues; increasing salivation; promoting gingival tissue regeneration; fostering osteogenesis in the boney structures of the teeth, mouth, and related areas; treating systemic diseases associated with oral bacteria; and treating other periodontal and oral maladies through the non-invasive application of weak direct current electricity to the surfaces in the oral cavity.

According to an aspect of an embodiment of a method according to the present invention, first and second electrodes are positioned in an oral cavity of a human. A plurality of electrokinetic elements, such as electrically charged (negatively or positively) charged elements (e.g., ions, charged molecules, charged molecular complexes) or polarized (e.g., dipole) or polarizable elements or agents are placed in the oral cavity. An electrical current is delivered between the first electrode and the second electrode, the electrical current causing movement of the electrokinetic elements at least one of across, into, and out of oral tissue of the human.

According to another aspect of an embodiment of a method according to the present invention, uncharged molecules may be suspended with the electrokinetic elements in a medium.

According to an aspect of an embodiment of a system according to the present invention, the system includes a mouthpiece sized and configured for placement in a mouth of a human and first and second electrodes supported by the mouthpiece. A plurality of electrokinetic elements (e.g., ions, charged molecules, charged molecular complexes) are disposed between the first electrode and the second electrode. An electrical current may be delivered between the first electrode and the second electrode so as to cause or enhance movement of at least some of the electrokinetic elements.

According to another aspect of an embodiment of a system according to the present invention, uncharged molecules may be suspended with the electrokinetic elements in a medium (e.g., a gel), which may include a whitening agent (e.g., for tooth and/or gingiva color lightening or brightening) and/or a dietary supplement, such as oil of oregano.

According to yet another aspect of an embodiment of a system according to the present invention, the electrokinetic elements may include one or more elements selected from the group consisting of an antibiotic, a probiotic, a prebiotic, an antifungal agent, an anesthetic, a growth factor, a chemotherapeutic, a monatomic ion, a monatomic ion complex, a polyatomic ion, a polyatomic ion complex, hydrogen peroxide, carbamide peroxide, an antiviral agent, a protein, an amino acid, a peptide, a polypeptide, urea, an anti-microbial enzyme, a vitamin, a mineral, insulin, nicotine, a salicylate, a salicylate derivative, an albitol (sugar alcohol), an amino sugar, a sugar substitute, a steroid, a classic eicosanoid, a non-classic eicosanoid, a cannabinoid, and a glycosaminoglycan.

According to an aspect of an embodiment of a device according to the present invention, the device includes a mouthpiece sized and configured to fit into a mouth of a human, and a plurality of electrodes supported by the mouthpiece, the electrodes being electrically coupled to a controller. Each electrode is programmable (preferably independently) as an anode or a cathode, or optionally removed from an electrical circuit altogether. A first electrode is positioned on an outer surface of the mouthpiece (further from a human cranial midline) and a second electrode is positioned on an inner surface of the mouthpiece (closer to the human cranial midline). The plurality of electrodes may include eight pairs of cooperating electrodes.

According to another aspect of an embodiment of a device according to the present invention, the mouthpiece further includes two opposing U-shaped channels configured to receive one or more teeth of the human. While electrodes may be independently programmable, all electrodes disposed on an outer surface of each U-shaped channel or both U-shaped channels may be the same polarity. Additionally or alternatively, all electrodes disposed on an inner surface of each U-shaped channel or both U-shaped channels may be the same polarity. Where a number of pairs of electrodes are provided, the same number of pairs of electrodes may be disposed along surfaces of each U-shaped channel.

According to an aspect of a method according to the present invention, a first electrode (which may be of a first polarity (anodic or cathodic)) may be positioned at a first location of gingival tissue of a human, the first location of gingival tissue at least partially surrounding at least one of (a) a tooth to be removed and replaced with an implant, (b) an empty tooth socket from which a tooth has been removed (accidentally or intentionally), and (c) a portion of a previously placed dental implant. A second electrode (which may be an opposite polarity to the first electrode) is positioned at a second location of gingival tissue of the human, the second location of gingival tissue at least partially surrounding at least one of (a) the tooth to be removed and replaced with an implant, (b) the empty tooth socket from which the tooth has been removed (accidentally or intentionally), and (c) the portion of the previously placed dental implant. Such positioning of the first and second electrodes may be achieved by supporting the electrodes on a mouthpiece, preferably in relatively static relation to each other (which relation or position may be customized to a particular individual). An electrical current is delivered between the first electrode and the second electrode. Such electrical current delivery may be achieved by electrically coupling the first electrode and the second electrode to a power supply and causing the power supply to discharge electrical current to one of the first electrode and second electrode.

According to another aspect of a method according to the present invention, the first location of gingival tissue comprises one of exterior gingival tissue (e.g., buccal, facial, or vestibular) and interior (e.g., lingual or palatal) gingival tissue, and the second location of gingival tissue comprises the other of the exterior gingival tissue and the interior gingival tissue.

According to still another aspect of a method according to the present invention, electrical current may be prevented from being delivered from or received by a third electrode, the third electrode being electrically isolated from the first electrode and the second electrode. Such prevention may be achieved by refraining from placing the third electrode in an oral cavity of the human, deactivating the third electrode, and/or electrically insulating the third electrode.

### BRIEF DESCRIPTION OF THE DRAWINGS

FIG. 1 is a perspective view of a treatment apparatus according to the present invention.

FIG. 2 is a top perspective view of a mouthpiece according to the present invention.

FIG. 3 is a bottom perspective view of a mouthpiece according to the present invention.

FIG. 4 is a front perspective view of a flat pattern according to the present invention.

FIG. 5 is a bottom perspective view of a flat pattern according to the present invention

FIG. 6 is a top plan view of a flex circuit according to the present invention.

FIG. 7 is a bottom plan view of a flex circuit according to the present invention.

FIG. 8 is a top perspective view of a bite plane according to the present invention.

FIG. 9 is a bottom perspective view of a bite plane according to the present invention.

FIG. 10 is a top plan view of a cable according to the present invention.

FIG. 11 is a front elevation view of a controller according to the present invention.

FIG. 12 is a bottom perspective view of the controller shown in FIG. 11.

FIG. 13 is a perspective view of a charging station according to the present invention.

FIG. 14 is an exploded perspective view of the charging station shown in FIG. 13.

### DETAILED DESCRIPTION

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

Preferred systems according the present invention preferably include the following:
(1) an electrical stimulus generator;
(2) at least one pair of electrodes (one electrode may include ground/Earth),
(3) charged molecule(s) intended to be transferred or to be collected (charged molecules intended to be collected need not be part of or supplied by the system itself);
(4) optionally a medium, gel, or solution between the active electrode and target tissue or fluid for charged particles to move through (saliva, blood, mucus, or other bodily tissue or fluids may serve as such).

Electrical stimulation parameters that may be programmed into, accessed by, or utilized by the generator may include the following:
predetermined stimulation duration of about 1 minute to a maximum of 72 hours depending on the treatment application, medication used, and electrode current density. More preferably, the stimulation duration is between about 10 minutes and about 30 minutes.
predetermined current intensity, which may be generally dependent on the surface area of the active electrode, which may range from 0.1 mA to 5 mA, preferably 1 mA to 5 mA, and more preferably from about 3 mA to about 5 mA.
predetermined medication dose, which may be determined by a function of the predetermined current intensity and predetermined stimulation duration such that total iontophoretic drug dose delivered (mA-min)=current intensity (mA)*treatment time (mins), which may range from 0 mA-min to 4320 mA-min and preferably from 0 mA-min to 400 mA-min, depending on the type of medication used and the current density of active electrodes.
predetermined electrical current density of the active electrode (or electrical current flux when pulsed DC stimulation is used), preferably from 0.1 mA/cm2 to 1.0 mA/cm2.
If pulsed DC stimulation is used (or optional AC stimulation) pulse frequencies ranging from about 5 kHz to about 80 kHz, and more preferably about 5 kHz to about 10 kHz.
An arbitrary waveform that has been uniquely optimized for:
Improved movement (larger quantity or shorter delivery duration) for a specific charged molecule
Better patient tolerability (less sensation or shorter delivery duration)

These effects are accomplished by the delivery of electrical current into the oral cavity (such as to the gingiva) through a plurality of electrodes in electrical contact (which may be direct physical contact) with gingival tissue surfaces (e.g., lingual, buccal, palatal, and/or vestibular gingival tissue), teeth, or other oral tissue. The electrodes may be fashioned out of any electrically-conductive material, including but not limited to metals such as silver, stainless steel, copper, gold, platinum, palladium, aluminum, an alloy thereof, electrically-conductive nanotubes, carbonized rubber, electrically-conductive silicone, or electrically-conductive polymers. The electrodes may be composed of the same or of differing materials. These electrodes fit snuggly against the lingual and buccal sides of the gingiva and make direct contact with each side of the gingiva to pass direct current electricity across the teeth and neighboring gingival tissues.

The electrodes on each side (lingual or buccal) of the gingiva may be of the same polarity. Electrodes on opposite sides of the gingiva may be of the opposite polarity. This allows the current to flow across the teeth and gums to the electrodes positioned on the transverse gingiva to complete the electrical circuit. Put another way, all electrodes on the lingual side of the gingiva may be completely anodic or completely cathodic. All electrodes on the buccal surfaces of the gingiva, transverse the lingual surfaces of the gingiva, may have the opposite polarity. The polarization of these electrodes may be reversed and/or alternated during treatment or in between treatments.

The mandibular and maxillary gingiva each may have a set of a plurality of polarized electrodes as previously described. This allows for treatment of both the maxillary and mandibular periodontium either simultaneously or in isolation. The maxillary and mandibular sets of electrodes may be powered by two different adjustable power supplies or by the same adjustable power supply.

Additionally or alternatively, the electrodes on each side (lingual or buccal) of the gingiva may be of different same polarity. Electrodes on opposite sides of the gingiva may be of the same polarity. This allows the current to flow along a particular side of the gums to the electrodes positioned on the same gingiva to complete the electrical circuit.

Electrical conductors then connect these electrodes to an adjustable power supply. All of the anodic electrodes will connect to the positive pole of the power supply and all of the cathodic electrodes will connect to the negative pole of the power supply.

In order to increase conductivity in the tissues adjacent to the electrodes, an ionic or colloidal liquid or gel may be used as a conductive medium to decrease electrical resistance in the mouth. This medium would be placed along any desired areas of desired electrical contact, such as the teeth, gums, or surrounding oral tissues. Examples of such a medium would include, but not be limited to, colloidal silver gel, liquid colloidal silver, colloidal copper gel, liquid colloidal copper, colloidal gold gel, liquid colloidal gold, saline gel, liquid saline or any combination thereof.

Colloidal silver, in whole or in combination, has great promise not only in increasing electrical current flow, but also in offering additional bactericidal benefits. Colloidal silver, in concentrations as little as five parts per million, is known to be bactericidal by inhibiting a bacterium's production of adenosine triphosphate.

This conductive medium may also contain dietary supplements including, but not limited to, oil of oregano. Oil of oregano is believed to have many health benefits and may also be microbicidal. Such microbicidal properties would be effective in treating common oral infections and diseases as well as aiding in preventative oral care.

This conductive medium may also contain teeth whitening agents. This would allow for the addition of teeth whitening to the list of benefits offered by an embodiment of this invention. A whitening agent that is catalyzed by direct current electricity could be included and may even offer reduced teeth whitening treatment times when compared with nonelectrically-catalyzed whitening agents.

Artificial or natural flavorings may also be added to this conductive medium to offer a more appealing taste to the user, similar to the method of flavoring dental fluoride treatments. This flavoring would mask any unpleasant tastes from the ingredients of the conductive medium or as well as any taste of the mouthpiece or electrodes themselves.

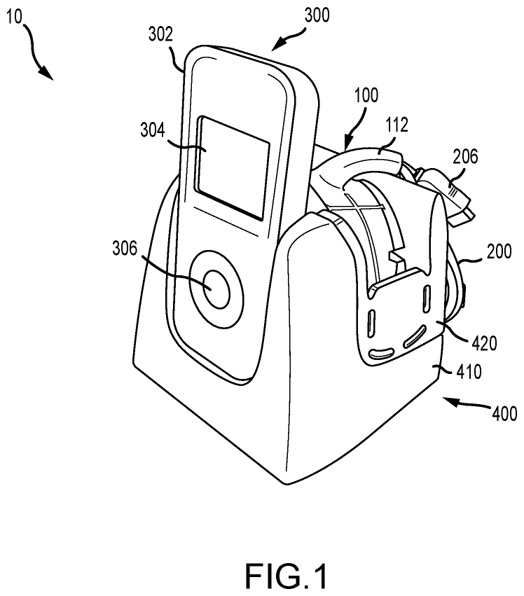

FIG. 1 shows one embodiment of a treatment apparatus 10 that may be used to implement methods according to this invention. The treatment apparatus 10 is preferably a stand-alone device comprising a mouthpiece 100, a controller 300, and a charging station 400.

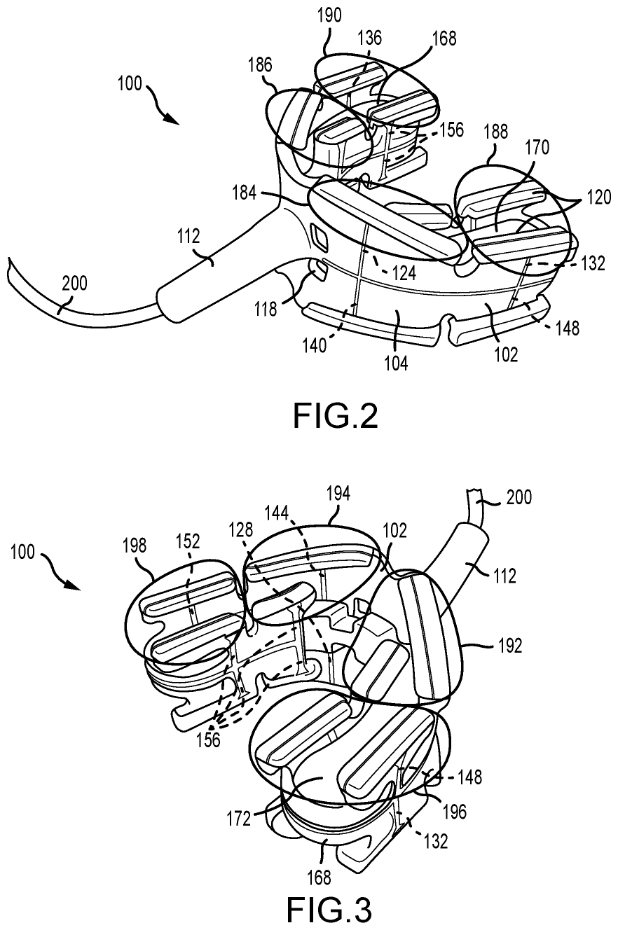

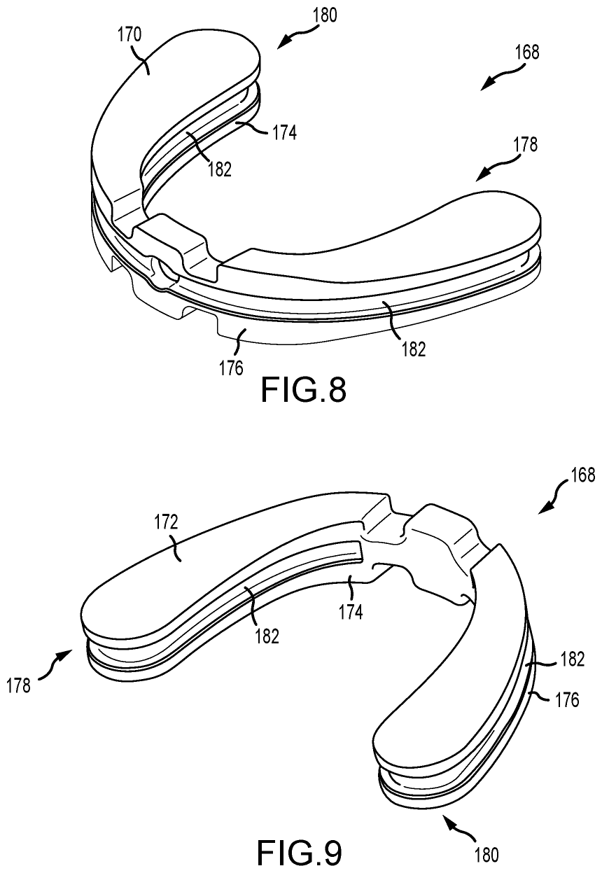

Looking at FIGS. 2 and 3, the mouthpiece 100 according to the present invention is shown. The mouthpiece 100 preferably comprises a flat pattern 102 and a bite plane 168 (see also FIGS. 8 and 9).

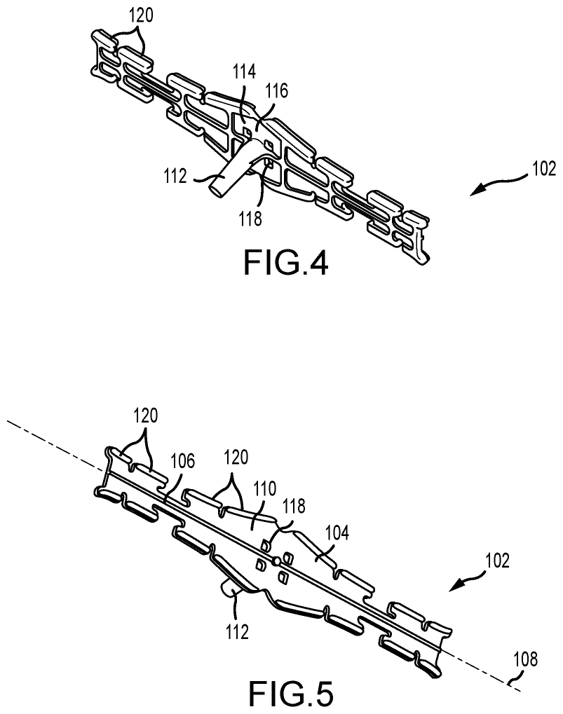

FIGS. 4 and 5 better show the flat pattern 102. The flat pattern 102 preferably comprises an encapsulant 104 encasing a flex circuit 122. Preferably the encapsulant 104 is a flexible polymer comprising a mixture of Silbione 4040AUI, Bluesil 4040 Activator and blue pigment. The flat pattern 102 preferably comprises a ridge 106 extending along a centerline 108 on an inside surface 110, a strain relief portion 112 extending outward from a center portion 116 of an outside surface 114, and a plurality of cutouts 118 extending from the inside surface 110 through the outside surface 114 approximate to the strain relief portion 112 configured to allow air passage to and from the user during use of the treatment apparatus 10. Preferably, the strain relief portion 112 curves downward when worn and can act as an indicator with respect to the proper orientation of the mouthpiece 100 during use.

The flat pattern 102 further comprises an electrically conductive polymer 120, preferably an electrically conductive silicone, provided at predetermined locations on the flat pattern 102 as discussed further below.

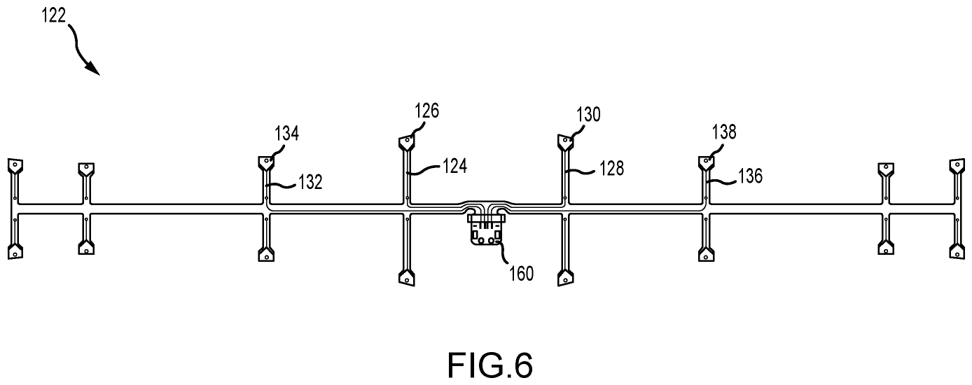

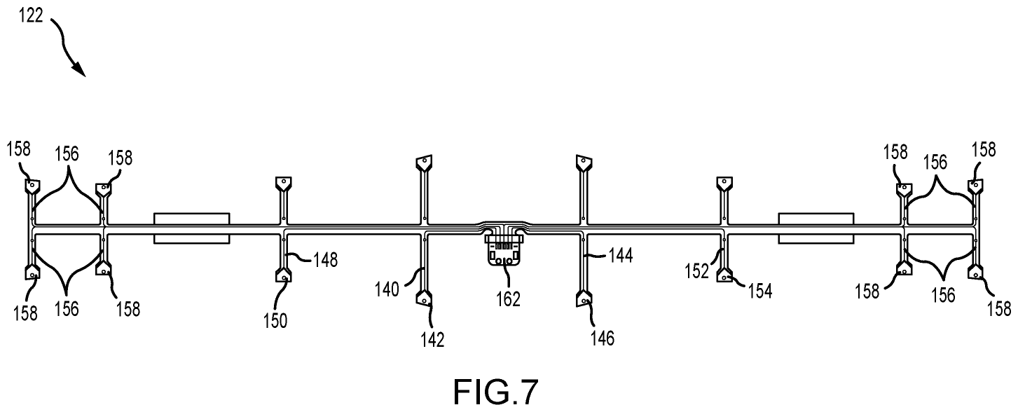

FIGS. 6 and 7 illustrate an exemplary embodiment of the flex circuit 122 according to the present invention. The flex circuit 122 is preferably formed from a copper-clad polyimide. The flex circuit 122 preferably comprises individual anodic electrodes 124, 128, 132, 136, 140, 144, 148, 152 (eight shown here) and a plurality of interconnected cathodic electrodes 156 (eight shown here) provided in a grid-like pattern.

It is preferable that the number of anodic electrodes is equal to the number of cathodic electrodes, but alternative arrangements are contemplated with different numbers of anodic and cathodic electrodes. Each anodic electrode 124, 128, 132, 136, 140, 144, 148, 152 has a distal end 126, 130, 134, 138, 142, 146, 150, 154, respectively, and each of the plurality of cathodic electrodes 156 has a distal end 158. Although shown as individual, controllable anodes and common cathodes, it is to be understood that targeted stimulation may be selectively provided, such as may be desirable to treat predetermined gingival areas. To provide targeted stimulation, delivery of electrical current to other portions of the mouth is preferably prevented or reduced mechanically or electrically. As an example, mechanical prevention or reduction may be achieved by particularized arrangement of electrodes, such as providing an anodic or cathodic electrode on a mouthpiece at a first location of gingival tissue that at least partially surrounds (a) a tooth to be removed and replaced with an implant, or (b) an empty tooth socket from which a tooth has already been removed intentionally or by accident, or (c) a portion of a previously placed dental implant. The mechanical prevention or reduction may be further enhanced by providing a cathodic or anodic electrode on the mouthpiece at a second location of (preferably on the opposite side of teeth from first location) gingival tissue that at least partially surrounds (a) the tooth to be removed and replaced with an implant, or (b) an empty tooth socket from which a tooth has already been removed intentionally or by accident, or (c) a portion of a previously placed dental implant. If the two electrodes are provided as described, and no other electrodes are disposed on the mouthpiece, then mechanical reduction of electrical current stimulation is achieved. In this way, a mouthpiece may be customized for a particular user by mechanically arranging electrodes on the mouthpiece to target electrical stimulation towards a dental implant site.

As an example of electrical prevention or reduction of non-targeted electrical current is selective electrode control by the controller. That is, mechanically there may be provided on a mouthpiece a plurality of electrodes spaced about the mouthpiece, as shown and described herein. However, through electrical control of such electrodes, each electrode may have a selectable state to provide stimulation. The selectable electrode state may be anodic, cathodic, or off (e.g., tri-stated). Thus, where targeted electrical current is desired, a first electrode on the mouthpiece may be selected to be an anodic or cathodic electrode. The first electrode position on the mouthpiece may correspond to a first location of gingival tissue that at least partially surrounds (a) a tooth to be removed and replaced with an implant, or (b) an empty tooth socket from which a tooth has already been removed intentionally or by accident, or (c) a portion of a previously placed dental implant. The electrical prevention or reduction of non-targeted electrical stimulation may be further enhanced by a first electrode on the mouthpiece being selected to be a cathodic or anodic electrode (opposite the first electrode). The second electrode position on the mouthpiece may correspond to a second location of (opposite side of teeth from first location) gingival tissue that at least partially surrounds (a) the tooth to be removed and replaced with an implant, or (b) an empty tooth socket from which a tooth has already been removed intentionally or by accident, or (c) a portion of a previously placed dental implant. If the two electrodes are selected as described, and no other electrodes are activated on the mouthpiece (e.g., all other electrodes are turned off or sent into a high impedance, or tri-state, mode), then electrical reduction of electrical current stimulation is achieved. In this way, a mouthpiece may be mechanically standardized for multiple users, but electrically customized to target electrical stimulation towards a dental implant site.

While mechanical and electrical prevention or reduction of stray or non-targeted electrical current has been described with respect to targeting a single dental implant site, it is to be understood that such targeting may be accomplished at multiple implant sites simultaneously or in a time sequenced fashion (e.g., one target site is stimulated for a predetermined time and then a different target site is stimulated for a predetermined amount of time).

FIG. 6 shows a top view of the flex circuit 122 wherein a first group of anodic electrodes (comprising anodic electrodes 124, 128, 132, 136) are each independently electrically connected to a first flat pattern connector 160. FIG. 7 illustrates a bottom view of the flex circuit 122 wherein a second group of anodic electrodes (comprising anodes 140, 144, 148, 152) are independently electrically connected to a second flat pattern connector 162 along with the plurality of cathodic electrodes 156. It should be noted that use of a single connector, in place of the first and second flat pattern connectors 160, 162, is also contemplated and should be understood to be within the purview of the present invention. The configuration described allows for each of the anodic electrodes 124, 128, 132, 136, 140, 144, 148, 152 to be independently energized. Though, as described above, every electrode on the mouthpiece may be programmable, in which case the common cathodic connection 156 would be replaced with separate connections to the connector 162. It is to be understood that the only exposed conductive surfaces on the flex circuit 122 are the distal ends of the electrodes and the electrical contacts on the connectors 160,162. The interconnections between the distal ends and the connectors are encased in a copper clad laminate, bondply or coverlay.

As stated above, the flex circuit 122, along with the first and second flat pattern connectors 160, 162, are preferably substantially encased in the encapsulant 104. The distal ends 126, 130, 134, 136, 140, 144, 148, 152 of the anodic electrodes 124, 128, 132, 136, 140, 144, 148, 152, respectively, and the distal ends 158 of each of the plurality of cathodic electrodes 156 are preferably coated with the conductive polymer 120 (see FIG. 5). Preferably, the conductive polymer 120 is exposed only on the inside surface 110 (i.e., the treatment side) of the flat pattern 102 and therefore is configured to not make unwanted contact with other tissues, like the lips, gums, and/or cheek of a patient.

The bite plane 168 is shown in greater detail in FIGS. 8 and 9. The bite plane 168 preferably comprises a flexible polymer, similar if not the same as the material comprising the flat pattern 102 (i.e., preferably a mixture of Silbione 4040AUI, Bluesil 4040 Activator and blue pigment), and is substantially u-shaped to follow the general teeth pattern of a human user. The bite plane 168 has a top surface 170, a bottom surface 172, an inside surface 174, an outside surface 176, a left portion 178, and a right portion 180. A groove 182 preferably extends along the outside surface 176 and a majority of the inside surface 174.

The groove 182 of the bite plane 168 is configured to receive the ridge 106 of the flat pattern 102 (see FIG. 5) and is preferably secured with an adhesive (not visible). Whereby, when assembled, the plurality of cathodic electrodes 156 is positioned proximate to the inside surface 174 of the bite plane 168, with distal ends 158 of the individual cathodic electrodes extending above the top surface 170 and below the bottom surface 172. The anodic electrodes 124, 128, 132, 136, 140, 144, 148, 152 are positioned proximate to the outside surface 176 with the distal ends 126, 130, 134, 138 above the top surface 170 and the distal ends 142, 146, 150, 154 below the bottom surface 172 of the bite plane 168 opposite a respective cathodic electrode of the plurality of cathodic electrodes 156. Preferably, the conductive polymer 120 is in contact with the distal ends 126, 130, 134, 138, 142, 146, 150, 154 of each of the anodic electrodes 124, 128, 132, 136, 140, 144, 148, 152 and the distal ends 158 of each of the plurality of cathodic electrodes 156, individually and separately. The flat pattern 102 is preferably sized and configured for the conductive polymer 120 to be positioned at or near the gingival margin (not shown), in physical contact with gingival tissue (and preferably not in physical contact with teeth) in the user's mouth (i.e., where the gums meet the surface of the teeth), whereby the anodic electrodes 124, 128, 132, 136, 140, 144, 148, 152 span the exterior (buccal and/or vestibular) gingival surfaces of the mouth and the plurality of cathodic electrodes 156 span the interior (lingual and/or buccal) gingival surfaces of the mouth, in this configuration.

The arrangement of opposing anodic and cathodic electrodes defines eight treatment zones (here shown as treatment zones 184, 186, 188, 190, 192, 194, 196, 198) which may be independently controlled as discussed further below.

Looking at FIG. 2 (and also FIG. 3 for reference) it is shown that treatment zone 184 comprises anodic electrode 124 and an opposing cathodic electrode of the plurality of cathodes 156, treatment zone 186 comprises anodic electrode 128 and an opposing cathodic electrode of the plurality of cathodes 156, treatment zone 188 comprises anodic electrode 132 and an opposing cathodic electrode of the plurality of cathodes 156, and treatment zone 190 comprises anodic electrode 136 and an opposing cathodic electrode of the plurality of cathodes 156.

Looking at FIG. 3 (and also FIG. 2 for further reference) it is shown that treatment zone 192 comprises anodic electrode 140 and an opposing cathodic electrode of the plurality of cathodes 156, treatment zone 194 comprises anodic electrode 144 and an opposing cathodic electrode of the plurality of cathodes 156, treatment zone 196 comprises anodic electrode 148 and an opposing cathodic electrode of the plurality of cathodes 156, and treatment zone 198 comprises anodic electrode 152 and an opposing cathodic electrode of the plurality of cathodes 156.

It is also contemplated that the electrode polarization of the mouthpiece 100 could be reversed at any time, even during the administration of treatment.

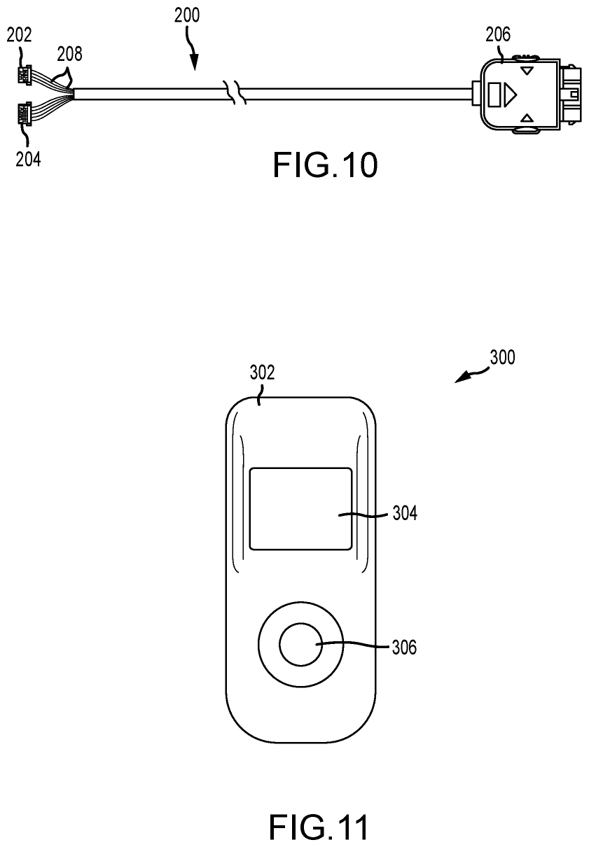

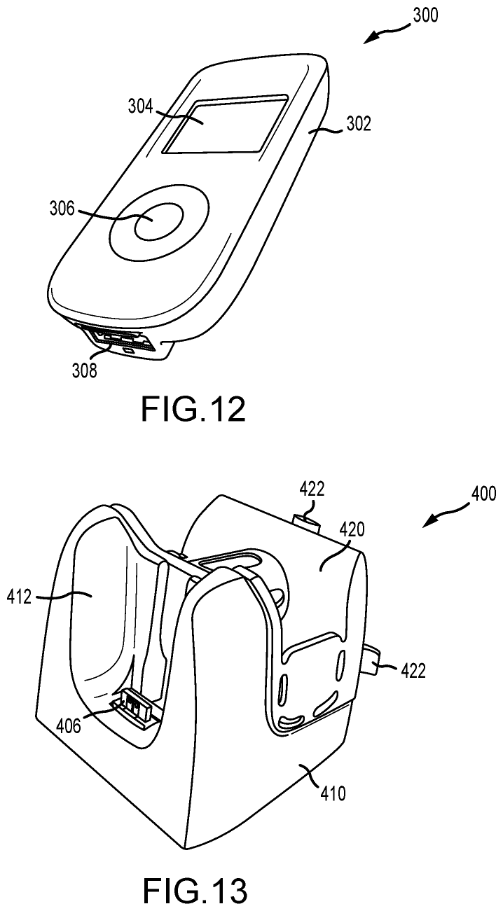

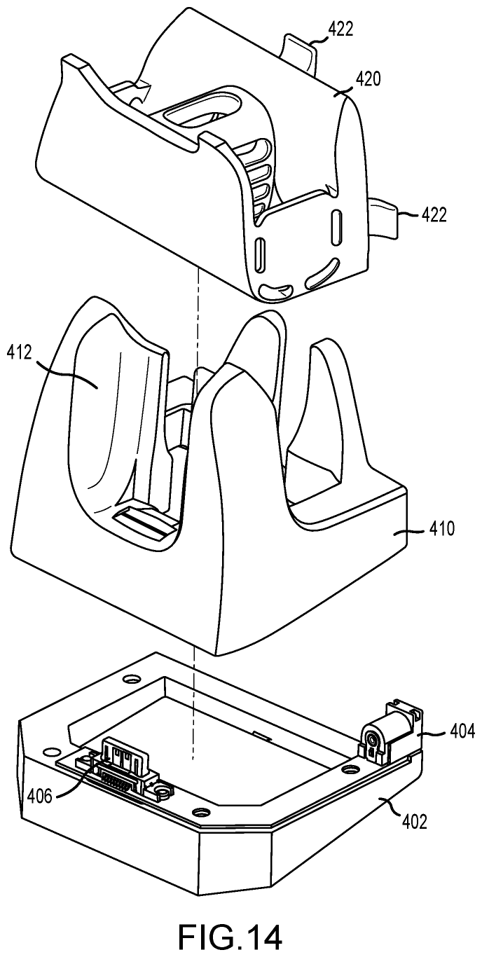

FIG. 10 shows an exemplary embodiment of a cable 200 according to the present invention. The cable preferably comprises a first cable connector 202 and a second cable connector 204, both electrically connected to a third cable connector 206 through a plurality of conductors 208 configured to be associated with each of the anodic electrodes 124, 128, 132, 136, 140, 144, 148, 152 and the plurality of cathodic electrodes 156. The first cable connector 202 is configured to interface with the first flat pattern connector 160 and the second cable connector 204 is configured to interface with the second flat pattern connector 162. One or several electrical conductors provided in the cable 200 may be jacketed with silicone rubber, and the cable 200, itself, is preferably a silicone jacketed cable.

The controller 300 preferably comprises a body 302, a liquid crystal display (LCD) screen 304, a push button 306, a controller connector 308, and a printed circuit board (PCB) (hidden). The controller 300 preferably delivers direct current of a predetermined amplitude and/or at a predetermined frequency. Pulsed bi-phasic current, alternating current, or other current may also be used.

The operation of the controller 300 by a user (not shown) is preferably performed through the pressing of the push button 306. For example, the user can start or pause the delivery of current to the mouthpiece 100 by pressing the push button 306. To prevent unintentional operation, the duration of the pressing of the push button 306 is sensed.

Depending on the state of the controller 300, a press of the push button 306 can have multiple functions. For example, in an exemplary embodiment, when in an off state, a press-and-hold of the push button 306 for approximately 1.5 seconds will turn on the controller 300 and further enter into a ready state. When in the ready state, a press-and-release of the push button 306 will enter the controller 300 into a run state and start the output of current to the mouthpiece 100. Whereas, a press-and-hold of the push button 306 for approximately 3.0 seconds when in the ready state will shut the controller 100 off. When in the run state, a press-and-release of the push button 306 will enter the controller 300 into a pause state and pause the output of current to the mouthpiece 100. A press-and-hold of the push button 306 for approximately 3.0 seconds when in the run state will shut off the controller 300. When in the pause state, a press-and-release of the push button will return the controller 300 to the run state, and a press-and-hold for 3.0 seconds when in the pause state will shut off the controller 300. After the treatment program has completed, the controller 300 will enter a complete state and a press-and-hold of the push button 306 will shut off the controller 300.

The LCD screen 304 preferably indicates the status of the treatment apparatus 10 to the user. For example, the LCD screen 304 may display indications such as, “Ready to Treat,” “Running,” “Check,” and “Fault.” “Ready to Treat” indicates that the controller 300 is in the ready state and ready to begin the delivery of direct current to the mouthpiece 100. “Running” means that the controller 300 is in the run state and delivering electrical current to the mouthpiece 100.

Preferably, when in the run state, a timer countdown depicting remaining treatment time may be output to the LCD screen 304. The LCD screen 304 may also preferably display the electrical current set point, indicating the amount of electrical current being delivered to the mouthpiece 100 and/or amount of current being sensed by the controller 300. Preferably, this status is displayed for approximately 5.0 seconds out of every 30 seconds, but may be displayed during the entire run state. “Check” may indicate that an open circuit has been detected. “Fault” may indicate an over-current fault and/or a low battery voltage condition.

Preferably, a light emitting diode (LED) (hidden) will illuminate when the controller 300 is in the run state. A tone generator (e.g., buzzer, speaker, etc.) (hidden) preferably delivers an audible tone to indicate a change in state and/or status and may provide feedback to the user for button presses and configuration events (discussed further below).

The controller 300 is preferably configured by a clinician or other trained staff member prior to a user interfacing with the treatment apparatus 10. Additionally, or alternatively, the patient may configure the controller 300. Preferably configuration of the controller 300 is performed through attachment of an additional piece of hardware (not shown) connected to the controller 300, but may also take place through a wireless connection (e.g., Bluetooth®, Wi-Fi, near field communications (NFC), infrared, magnetic). Finally, the controller 300 may be provided with a default treatment regimen to reduce or eliminate initial configuration effort by a clinician or patient.

Configuration parameters preferably include: selection of any combination of the treatment zones 184, 186, 188, 190, 192, 194, 196, 198 to provide direct current for treatment; selection of direct current output values, for example, 6 μA, 12 μA, 18 μA, 25 μA, 50 μA, 62 μA, 75 μA, 100 μA, 125 μA, 150 μA, and 200 μA (preferably not to exceed 1,000 μA total current across all treatment zones at any one time); and selection of treatment duration (preferably from 1 minute through 30 minutes selectable in increments of 1 minute).

The controller 300 is preferably capable of monitoring compliance of treatments performed by the treatment apparatus 10 and recording a number of performance metrics on an electrical erasable programmable read-only memory (EEPROM). The controller connector 308 is preferably configured to be compatible with the JTAG (Joint Test Action Group) standard to aid in accessing the EEPROM and the compliance records by a computer or other electronic device (not shown). The records may be utilized by a clinician (not shown) to evaluate and discuss the treatment.

For example, some metrics and data collected may include the following (along with the dates and times of such occurrences): the number of treatments started; the number of successfully completed treatments; the number of open circuit faults; the number of treatments with an open circuit; the number of treatments with an open circuit that still completed treatment successfully; the number of overcurrent faults; the number of low battery faults; the number of times the device was paused; the number of treatments that were paused but still completed treatment successfully; the number of times the user turns the device on; the number of times the device is powered off by the user; the number of times the device is powered off by software; and the total number of minutes the device has run since a memory reset.

The controller 300 may also be configured to dynamically monitor the electrical characteristics (i.e., resistance, voltage, current) of each treatment zone and adjust the treatment without clinician or user intervention. For example, if one of the anodic electrodes 124, 128, 132, 136, 140, 144, 148, 152 makes contact with one of the plurality of cathodic electrodes 156 through a metal filling or crown in the mouth of a patient and therefore completely bypasses the gingiva to be treated, the controller 300 may be configured to detect the artificially low resistance in the return current and disable the affected treatment zone.

A real-time clock (hidden) is also preferably included to record the time and date at which the metrics and data are collected.

The EEPROM will preferably store the following information about a currently running treatment: how many minutes of treatment (up to 30 minutes) the user completed of treatment; whether the mouthpiece was disconnected while running; whether an overcurrent fault occurred; and whether a low battery fault occurred; how many pauses the user initiated during treatment; and how many open circuit checks occur during the treatment.

It is also contemplated that the controller 300 may be configured to detect when the mouthpiece 100 is disconnected from the controller 300. This may be accomplished through a test of continuity between two pins on the third cable connector 206 of the cable 200 and the controller connector 308.

The controller 300 may also be configured to detect when, although there is a connection between the mouthpiece 100 and the controller 300, the mouthpiece 100 is not located in the mouth of a user. To do so, the controller 300 monitors the delivery of current and whether current is detected on any of the plurality of cathodic electrodes 156 (i.e., the return path). If no current is detected on the return path, the controller 300 pauses treatment and indicates an error on the LCD screen 304. For instance, the controller 300 monitors the stimulation circuits, including the anodic and cathodic electrodes. The controller 300 includes circuitry to measure or predict the amount of current to be delivered to the mouthpiece 100 (delivered current), and also to measure the amount of return current received from the mouthpiece 100 (return current). The circuitry then compares the return current to the delivery current, and if the difference is greater than a predetermined value (e.g., a percentage of the delivered current, such as 10% to about 50%), then stimulation is paused, preferably on all electrodes, and a fault message is displayed on the controller. Once the difference between delivered current and return current is less than the predetermined amount, then the stimulation program or regimen will resume from where it left off, preferably so no or little treatment time is lost.

Graph 1 below provides parameters for monitoring the current delivered to a treatment zone. As described above, the running current setpoint and also the duration “T” is determined and set during configuration prior to a patient using the treatment apparatus 10, with the recommended setting for duration “T” at two seconds. The open circuit is preferably approximately 80% of the running current set point and the over current fault limit is preferably 120% of the running current set point. The hardware limit is preferably approximately 200 to 300 μA per stimulation channel (e.g., per anodic electrode).

The current on the return path is preferably polled eight times per second when the controller 300 is delivering current to any of the treatment zones.

If the current detected on the return path is less than the open circuit limit in any of the treatment zones for more than the preset duration, all of the problematic treatment zones will pause and a notification will be displayed on the LCD screen 304 to check the mouthpiece 100. Additionally, or alternatively, when the current detected on the return path is less than the open circuit limit in any of the treatment zones for more than the preset duration, all treatment zones will pause and a notification will be displayed.

If the current detected on the return path is more than the over current fault limit in any of the treatment zones for more than the preset duration, current will be stopped to all treatment zones and the LCD screen 304 will display a fault notification.

It is further contemplated that the treatment apparatus 10 be fully compatible with wireless technology such as Bluetooth® technology, near-field communication, and wi-fi to communicate with a user's electronic device (not shown), such as a cell phone, tablet, or personal computer. Preferably, a user may review usage history, the prescribed treatment plan, and/or a comparison of usage history versus treatment plan. The treatment apparatus 10 may also provide notifications regarding scheduled treatment sessions to any of the user's electronic devices. This functionality is contemplated as operating through an application (not shown) downloadable to a user's electronic device. The application may also be configured to share this data with a central server for storage, remote monitoring by the prescribing clinician, provide one-way or two-way communication between patient and clinician, and/or allow for a clinician to remotely adjust the treatment parameters. Additionally, firmware upgrades may be supplied to the controller 300 wirelessly.

Charging station 400 preferably comprises a base 402, a cradle 410, and a mouthpiece cup 420. The base 402 preferably comprises a power input 404 and base connector 406. The power input 404 is configured to receive input power from a power input source (not shown) (for example, a direct current transformer plugged into a standard electrical outlet providing alternating current). The base connector 406 is preferably configured to be received within the controller connector 308 and deliver power to a rechargeable power source (not shown) within the controller 300.

The cradle 410 is configured to be coupled with the base 402 and to removably receive the mouthpiece cup 420. The cradle 410 preferably has a pocket 412 sized and configured to removably receive the controller 300.

The mouthpiece cup 420 is preferably configured to hold the mouthpiece 100 and has a plurality of protrusions 422 around which the cable 200 may be wrapped.

In another embodiment of this invention or in combination with those previously described, an ionic or colloidal medium in the form of a liquid or a gel may be used to decrease electrical resistance in the mouth and to facilitate a more even current distribution across oral electrodes. Any combination of one or more ionic or colloidal compounds may be used. Examples of such a medium would include, but not be limited to, colloidal silver gel, liquid colloidal silver, colloidal copper gel, liquid colloidal copper, colloidal gold gel, liquid colloidal gold, saline gel, liquid saline or any combination thereof. Artificial or natural flavorings may be added to this medium to offer a more appealing taste to the user. The medium may also contain dietary supplements including, but not limited to, oil of oregano. This medium may also contain teeth-whitening chemical agents. A whitening agent that is catalyzed by the direct current would be most effective in this ionic or colloidal medium.

Thus, at least one embodiment addresses a desired need in the oral hygiene and dental fields to concurrently treat common oral diseases and conditions in a more effective, less invasive, and less expensive manner. These embodiments promote general oral hygiene, reduce oral biofilm, treat periodontal diseases such as gingivitis and periodontitis, kill oral microbes including bacteria and thus preventing cavities and tooth decay, increase vasodilation and blood flow in oral tissues, promote gingival tissue regeneration, foster osteogenesis in the boney structures of the teeth, mouth, and related areas, treat systemic diseases related to oral pathogens, and treat other periodontal and oral maladies through the non-invasive application of weak direct current electricity to the surfaces in the oral cavity.

In some cases, dental procedures can break up oral bacterial colonies found in biofilms and introduce bacteria into the bloodstream causing bacteremia and other infections. It is further contemplated that it may be desirable to utilize a mouthpiece according to the present invention immediately prior to performing a dental procedure. The treatment apparatus 10 according to the invention may be used by the patient either at home or in the dental office. In this manner, the living bacteria in the patient's mouth, both supra- and sub-gingival, can be reduced prior to the procedure and the risk of bacteremia and other infections will be reduced. For example, and not by way of limitation, the treatment apparatus 10 may be utilized prior to a dental prophylaxis or a scaling and root planning procedure in a dental office to reduce the risks of introducing bacteria into the patient's blood stream.

The treatment apparatus 10 may also be utilized following a clinical procedure as prevention for infections, for scenarios including but not limited to post-extraction or post-implantation infection prevention.

These treatments for biofilm reduction and prevention may be repeated on a daily basis for three to six weeks for acute biofilm-based issues or may be repeated once or more per week on a continuing basis for chronic biofilm issues.

Treatment and/or Prevention of Peri-Implantitis

Peri-implantitis is generally inflammation of oral tissue in physical contact with, surrounding, or otherwise in proximity to, and occurring after, placement of a dental implant. This inflammation may be reduced or prevented using methods according to the present invention. Methods may be performed before and/or after a dental implant surgical procedure of dental implant placement or replacement.

A method of reducing a likelihood of peri-implantitis involves, prior to a dental implant being placed or replaced partially or in its entirety, applying or directing electrical current to gingiva tissue near or at an oral site of future implantation. While electrical current may be distributed elsewhere throughout oral tissue, at least 6 μA and more preferably at least approximately 50 μA of electrical current (and preferably no more than 300 μA) is delivered to the gingiva tissue near or at a predetermined oral site of future implantation. A pre-surgery treatment regimen may consist of approximately twenty minutes of electrical stimulation per day for one to fourteen days prior to a dental implant surgical procedure.

A method of reducing a likelihood of peri-implantitis (if it has not yet begun) or reducing peri-implantitis (if it has already begun) involves, after a dental implant has been placed or replaced partially or in its entirety, applying or directing electrical current to gingiva tissue near or at an oral site of implantation. While electrical current may be distributed elsewhere throughout oral tissue, at least 6 μA and more preferably at least approximately 50 μA of electrical current (and preferably no more than 300 μA) is delivered to the gingiva tissue near or at a predetermined oral site of implantation. A post-surgery treatment regimen may consist of approximately twenty minutes of electrical stimulation per day for one to fourteen days after a dental implant surgical procedure, or until desired inflammation reduction has occurred.

While the pre-surgery and post-surgery methods have been separately described for clarity, it is to be understood that either or (preferably) both methods may be utilized for a particular patient, or user of the mouthpiece.

### Iontophoresis/Reverse-Iontophoresis

Since the transport and movement of charged molecules within an electric field relies on a continuous driving force, iontophoresis and reverse iontophoresis systems generally rely on DC stimulation, which provides an uninterrupted, unidirectional, monophasic electric current to induce movement of ions. Pulsatile DC currents can be used in which a series of unidirectional, monophasic electric currents are delivered over short periods of time at regular intervals to inducement movement of ions similar to continuous DC stimulation.

AC stimulation, which is biphasic, may have a more limited ability to induce the movement of ions when a symmetric biphasic waveform is used, due to equal net charge displacement in each direction; however, AC stimulation can be employed with asymmetric and unbalanced waveforms that yield a greater net charge on one phase that can result in the movement of ions. It should be noted that an asymmetric and unbalanced biphasic current that results in a net charge greater than zero is considered to be a DC current by definition. Similarly, a symmetric biphasic AC current with a “DC offset” has the net effect of resulting in a unidirectional monophasic electric current equivalent to a DC current without biphasic AC components.

Methods according to the present invention may be employed using a variety of electrodes, charged molecules, stimulus parameters, and potential applications, there is a high degree of potential combinations of system and control elements that may achieve a specific therapeutic goal. Given a therapeutic goal and a set of safety limitations, any given embodiment may alter the operating characteristics of one or more system and control elements within the ranges described such that optimal treatment delivery may be achieved while preserving and/or enhancing patient safety.

At minimum at least one electrode (with the ground/Earth acting as a second electrode), but preferably at least one pair of discrete electrodes, is required to complete an electric circuit that includes at least a portion of oral tissue or mucous, although a plurality of electrodes can be used simultaneously and in concert or successively. The electrical polarity of each electrode may be changed during the treatment or between treatments to improve delivery or therapy.

Based on the therapeutic application, electrode configurations may be (i) monopolar, in which the active electrode is located at the target area and the inactive electrode located at nontreatment area (such as the hand, wrist, or foot), (ii) bipolar, in which both or all electrodes are located at target areas, (iii) multipolar, in which three or more electrodes of multiple circuits are located over target areas. One or more electrodes may be placed on or in electrical communication with any surface of the oral cavity, including both hard and soft tissues which may include the palate, tongue, sublingual mucosa, lingual mucosa, facial mucosa, and buccal mucosa. One, many, or all electrodes may also be located on the external surfaces of the body, preferably the head including the face, cheek, neck, lips, etc. with the intent that the current passes through such tissue into the oral cavity.

Embodiments employing monopolar configurations may position the inactive electrode in a remote location on the body from the active electrode, such as in the form of a finger or lip-clip, or in a concentric geometry with the active electrode as surface-adhesion patches in which both electrodes are in the shape of a ring and the active electrode is encompassed by the inactive electrode. Embodiments employing bipolar configurations may be effective even though the target tissue lies between the electrode and the medium/solution through which the charged molecule moves; these embodiments may employ 2×1 electrode configurations in which the inactive electrode is situated centrally between two active electrodes as well. Embodiments employing bipolar configurations may also be placed topically on tissues of the mouth. Furthermore, embodiments with bipolar electrode configurations can be envisioned such that can also be achieved, either specifically or simultaneously with other target tissues.

Embodiments employing multipolar configurations may utilize two or more active-inactive electrode pairs or multiple electrodes in N×1 arrangements of active-inactive electrodes (where N is at least 3) in such a fashion that enhanced control over the 3-dimensional movement of charged molecular may be achieved. In addition, embodiments employing multipolar configurations may utilize two pairs of active-inactive electrodes with one pair within the electrodes of the second pair; such a configuration could function simultaneously to generate an electric field as well as a tetrapolar bioimpedance analyzer to detect changes in the impedance of the target tissue and charged molecules within the medium; detection of these changes can subsequently be used as a control routine to regulate the stimulation parameters such that treatment automatically stops when the desired degree of charged molecule transfer has been achieved or upregulate stimulation parameters dynamically to maintain expected performance.

Further embodiments may include a reservoir, including charged molecules and/or medium, gel, or solution through which charged molecules will transfer can be supplied or generated by multiple means; as a result, a reservoir for the purposes of containing the ions or charged molecule(s) to be transferred or containing charged molecules that have been collected is optional. Embodiments with reservoirs may have a pre-loaded reservoir that is disposable or a reservoir that can be filled prior to use, as well as a reservoir that may be either pre-loaded or filled prior to use and also re-loaded for multiple uses. A reservoir may also be integrated into the electrodes or housed or supported by a mouthpiece such that either biological fluids or fluids that can be manually replenished may be used as the medium for charged molecules to move through. One possible embodiment of a reservoir integrated into the electrodes consist of disposable woven electroconductive fiber electrodes that capture charge molecules within the fibers and can be detached for analysis ex situ; similarly, “cages” composed of structured nanomaterial can be utilized for selective channeling of medium as well as direct capture of charged molecules.

Table of Agents & Indications

| Agent or Class/Family of | |
|---|---|
| Compound (i.e., | |
| electrokinetic elements | |
| and/or delivery media) | Purpose |
| Antibiotics (specifically | Reduce bacteria levels |
| those intended to reduce | associated with: |
| various oral infections | periodontal disease, |
| (e.g. Arestin, Atridox, | gingivitis, halitosis, |
| PerioChip, PerioStat, | tooth Decay, tooth |
| etc.); designed to be | Remineralization, peri- |
| inserted into subgingival | implant disease, oral |
| spaces, for topical use, | mucositis. |
| or use via injection; | Reduce gingival bleeding |
| administered as a | and inflammation of the |
| mouthwash, lozenge, or | oral tissues. |
| gum) including but not | Reduce the risk of |
| limited to chlorhexidine, | continued oral tissue |
| cetylpyridinium chloride, | loss. |
| benzalonium minocycline, | |
| doxycycline, etc. and | |
| their various | |
| preparations. | |
| Probiotic lozenges, | |
| including those that | |
| include Lactobacillus | |
| Antifungal Agents | Reduce levels of oral |
| (including azole | fungi to reduce the risk |
| compounds like | of infections such as |
| fluconazole, | Thrush and Candidiasis |
| ketoconazole, and | |
| triazoles) | |
| Local anesthetics and | Anesthesia |
| compounds regularly | |
| administered alongside | |
| local anesthetics, e.g. | |
| Aminobenzoic esters, such | |
| as benzocaine and | |
| procaine; Benzoic esters | |
| such as cocaine; Amides | |
| such as bupivacaine, | |
| lidocaine, mepivacaine, | |
| and prilocaine; | |
| Combinations of local | |
| anesthetics; Compounds | |
| regularly administered | |
| alongside local | |
| anesthetics such as | |
| epinephrine, | |
| levonordefrin, and other | |
| ion channel modulators, | |
| such as dihydropyridines | |
| and derivatives, | |
| diltiazem and | |
| derivatives, and | |
| gabapentinoids. Local | |
| anesthetics and compounds | |
| administered alongside | |
| local anesthetics may | |
| also include those that | |
| are unlisted but known to | |
| those skilled in the art. | |
| Growth stimulating | Stimulate cellular |
| compounds (“growth | proliferation and |
| factors”), including but | cellular differentiation |
| not limited to bone | for the purpose of bone |
| morphogenetic proteins | growth, vasculogenesis, |
| (BMPs), colony- | angiogenesis, and wound |
| stimulating factors, | healing |
| epidermal growth factors, | |
| ephrins, fibroblast | |
| growth factors, insulin- | |
| like growth factors, | |
| interleukins, | |
| keratinocyte growth | |
| factor, migration- | |
| stimulation growth | |
| factor, macrophage | |
| stimulation protein, | |
| transforming growth | |
| factors (TGF-α, TGF-β), | |
| tumor necrosis factor- | |
| alpha (TNF-α), and | |
| Vascular endothelial | |
| growth factor (VEGF), | |
| either individually or in | |
| combination, such as | |
| enamel matrix derivative | |
| or similar powders | |
| including but not limited | |
| to Pepgen P-15 | |
| Chemotherapeutics | Monotherapy or |
| including by not limited | combinational therapy |
| to nucleic acids, | treatment of cancer, |
| glycosylamines, related | tumors, and/or lesions |
| analogues and/or produgs; | |
| ionophores; | |
| photosensitizers | |
| Molecular ions, e.g. | Tooth remineralization |
| monoatomic ions, such as | |
| fluoride ion, and | |
| monoatomic ion complexes; | |
| polyatomic ions and | |
| polyatomic ion complexes, | |
| such as oxocarbon anions, | |
| chlorine oxyanions, | |
| phosphites, phosphates, | |
| sulfites, sulfates, | |
| nitrates, nitrites. Other | |
| monoatomic ions, | |
| monoatomic ion complexes, | |
| polyatomic ions, and | |
| polyatomic ion complexes | |
| may also include those | |
| known to those skilled in | |
| the art. | |
| Hydrogen peroxide and | Tooth whitening; |
| carbamide peroxide for | Antimicrobial effects |
| whitening and anti- | |
| microbial effects | |
| Antiviral agents | Local and systemic |
| including but not limited | treatment of viral |
| to vaccines; nucleic | infections; Prevention of |
| acids, glycosylamines, | viral infection |
| related analogues and/or | |
| produgs (such as | |
| Remdesivir and | |
| Favipiravir); ionophores | |
| (including quinazoloine, | |
| quinoline, and related | |
| derivatives, such as | |
| quinine, chloroquine, and | |
| hydroxychloroquine); | |
| lactones (such as | |
| Ivermectin); protease | |
| inhibitors (such as | |
| Lopinavir and Ritonavir). | |
| Amino acids (such as | Antibiotic and |
| arginine) Proteins, | Antimicrobial effects; |
| peptides (including | Growth Stimulating |
| cytokines, antimicrobial | Effects; Wound |
| peptides, and | Healing/Regeneration |
| glycoproteins such as | effects; Complement |
| immunoglobulins), and | System modulators for the |
| polypeptides (including | treatment of ischaemic, |
| glycoproteins such as | autoimmune, and |
| fibronectin) | inflammatory diseases |
| Penetration enhancers | Enhance penetration and |
| (including lipid | extraction of molecules |
| modifiers, protein | of interest |
| modifiers, and | |
| partitioning promoters) | |
| pH balancing and | Tooth demineralization |
| conditioning agents | prevention; Prevention of |
| including but not limited | adverse pH shifts during |
| to urea in combination | electrical stimulation |
| with arginine | |
| Anti-microbial enzymes | Anti-plaque and anti- |
| | biotic effects |
| Vitamins and minerals | For nutrition, oral |
| such as ascorbic acid; | wellness, and systemic |
| thiamine, riboflavin, | wellness |
| niacin and nicotinamide | |
| derivatives; pantothenic | |
| acid; pyridoxine and | |
| derivatives; biotin, | |
| folate; cobalamins, such | |
| as cyanocobalamin and | |
| methylcobalamin; fat- | |
| soluble secosteroids, | |
| such as cholecalciferol | |
| and ergocalciferol. Other | |
| vitamins may also include | |
| those known to those | |
| skilled in the art. | |
| Agents that help balance | General oral health |
| oral flora/microbiome | improvement |
| like pre-biotics | |
| Insulin | Management of blood sugar |
| | for diabetics |
| Nicotine | Reduction of nicotine |
| | dependence; Smoking |
| | cessation aid |
| Salicylates or | Analgesia; Anti- |
| derivatives, e.g. | inflammation; |
| salicylic acid, | Bacteriostatic and |
| diflunisal, and | fungicidal properties; |
| salsalate; acetyl | Keratolytic properties; |
| salicylates, such as | |
| aspirin; metal-salicylate | |
| complexes such as | |
| magnesium salicylate and | |
| bismuth subsalicylate; | |
| Quaternaries complexed | |
| with salicylates, such as | |
| choline salicylate. Other | |
| derivatives may also | |
| include other reactive | |
| groups known to those | |
| skilled in the art. | |
| Carbohydrates and | Immune system modulation; |
| carbohydrate | Prebiotic and probiotic |
| macromolecules including | effects; Anti-biotic |
| but not limited to | effects |
| disaccharides, | |
| oligosaccharides, | |
| polysaccharides, and | |
| saccharide macrocycles | |
| Sugar alcohols and sugar | Anti-microbial effects |
| substitutes (including | |
| sugar alcohols, such as | |
| xylitol, erythritol, | |
| mannitol, and sorbital, | |
| as well as aspartame and | |
| saccharin) and amino | |
| sugars (such as | |
| glucosamine) | |
| Steroids such as sex | Hormonal modification; |
| hormones, | Regulation of metabolism |
| corticosteroids, and | and immune function; |
| anabolic steroids | Modulation of salt and |
| including but not limited | water balance. |
| to dexamethasone, | |
| prednisone, | |
| hydrocortisone. | |
| Classic and non-classic | Anti-inflanimation; |
| eicosanoids and | Modification of immune |
| cannabinoids (both | function; Analgesia |
| endocannabinoids and | |
| phytocannabinoids) | |
| Glycosaminoglycans, | Biodegradable |
| including but not limited | microneedles to enhance |
| to hyaluronic acid | therapy |
| Heterogenous mixtures of | Conditioning target |
| the above with or without | tissue or relevant |
| non-bioactive co-agents | microenvironments to |
| | optimize treatment |
| | delivery and/or achieve |
| | simultaneous, |
| | multidirectional delivery |
| | of bioactive agents; |
| | Anti-microbial, Anti- |
| | Fungal, Anti-viral |
| | effects; Chemotherapy and |
| | Photoimmunotherapy; |
| | Photodynamic Therapy |
| | (including antimicrobial |
| | photodynamic therapy and |
| | “vascular targeted” |
| | photodynamic therapy |
| Agents that may be | Introduction of agents |
| delivered sublingually | into blood and tissues in |
| (under the tongue), | such a way to avoid |
| including but not limited | first-pass metabolism in |
| to cardiovascular drugs, | the liver |
| steroids, barbiturates, | |
| benzodiazepines, opioid | |
| analgesics, THC, CBD, | |
| enzymes, Suboxone, | |
| vaccines, and therapeutic | |
| peptides. | |

Electrokinetic elements, as used herein, should be understood to reference particles and/or fluids (or combinations thereof) that have a net ionic charge, consist of or include electrical dipoles, and/or are otherwise polarizable by an electric field. In performing methods in accord herewith, electrokinetic elements are introduced or delivered into an oral cavity of a human by a delivery method. At a random or predetermined time thereafter, electrical stimulation is delivered as described herein, causing movement of the electrokinetic elements, thereby preferably causing, improving or adjunctively assisting absorption, desorption, or adsorption of predetermined agents or compounds into, out of, or across/onto oral tissue. Such element delivery methods, as indicated above, may include mouthwash, lozenge, paste, gel, subgingival and/or intragingival injection, sublingual and/or intralingual injection, subpalatal and/or intrapalatal injection, atomization, fluid dropper, and/or other method of depositing the selected element in a selected area of an oral cavity.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is described by the claims.

What is claimed is:

1. A method comprising the steps of:
positioning a first electrode in an oral cavity of a human;
positioning a second electrode in the oral cavity;
placing a plurality of electrokinetic elements in the oral cavity;
delivering an electrical current between the first electrode and the second electrode,
wherein the electrical current causes movement of the electrokinetic elements at least one of across, into, and out of gingival tissue of the human.

2. The method according to claim 1, wherein the electrokinetic elements comprise ions.

3. The method according to claim 1, wherein the electrokinetic elements comprise charged molecules.

4. The method according to claim 1, wherein the electrokinetic elements comprise charged molecular complexes.

5. The method according to claim 1, further comprising uncharged molecules suspended with the electrokinetic elements in a medium.

6. A system comprising:
a mouthpiece sized and configured for placement in a mouth of a human;
a first electrode supported by the mouthpiece;
a second electrode supported by the mouthpiece;
a plurality of electrokinetic elements disposed between the first electrode and the second electrode,
wherein, an electrical current delivered between the first electrode and the second electrode causes movement of at least some of the electrokinetic elements at least one of across, into, and out of gingival tissue of the human.

7. The system according to claim 6, wherein the electrokinetic elements comprise ions.

8. The system according to claim 6, wherein the electrokinetic elements comprise charged molecules.

9. The system according to claim 6, wherein the electrokinetic elements comprise charged molecular complexes.

10. The system according to claim 6, further comprising uncharged molecules suspended with the electrokinetic elements in a medium.

11. The system according to claim 10, the medium comprising a gel.

12. The system according to claim 10, the medium comprising a dietary supplement.

13. The system according to claim 12, the dietary supplement comprising oil of oregano.

14. The system according to claim 10, the medium comprising a whitening agent.

15. The system according to claim 6, the electrokinetic elements comprising one or more elements selected from the group consisting of an antibiotic, a probiotic, a prebiotic, an antifungal agent, an anesthetic, a growth factor, a chemotherapeutic, a monatomic ion, a monatomic ion complex, a polyatomic ion, a polyatomic ion complex, hydrogen peroxide, carbamide peroxide, an antiviral agent, a protein, an amino acid, a peptide, a polypeptide, urea, an anti-microbial enzyme, a vitamin, a mineral, insulin, nicotine, a salicylate, a salicylate derivative, an albitol (sugar alcohol), an amino sugar, a sugar substitute, a steroid, a classic eicosanoid, a non-classic eicosanoid, a cannabinoid, and a glycosaminoglycan.

16. A device comprising:
a mouthpiece sized and configured to fit into a mouth of a human; and
a plurality of electrodes being electrically coupled to a controller and being supported by the mouthpiece, each electrode comprising one of an anodic electrode and a cathodic electrode, at least a first electrode disposed on an outer surface of the mouthpiece and a second electrode being disposed on an inner surface of the mouthpiece;
wherein each electrode is selectively programmable as an anode or a cathode by the controller separate and apart from any other electrode on the mouthpiece.

17. The device according to claim 16, the mouthpiece further comprising: two opposing U-shaped channels configured to receive one or more teeth of the human.

18. The device according to claim 17, wherein all electrodes disposed on an outer surface of each U-shaped channel are the same polarity.

19. The device according to claim 17, wherein all electrodes disposed on an outer surface of both U-shaped channels are the same polarity.

20. The device according to claim 17, where all electrodes disposed on an inner surface of each U-shaped channel are the same polarity.

21. The device according to claim 17, wherein all electrodes disposed on an inner surface of both U-shaped channels are the same polarity.

22. The device according to claim 17, wherein the plurality comprises eight pairs of electrodes.

23. The device according to claim 22, wherein four pairs are disposed along surfaces of each U-shaped channel.

24. A method comprising the steps of:
positioning a first electrode on a mouthpiece at a first location of gingival tissue of a human, the first location of gingival tissue at least partially surrounding at least one of (a) a tooth to be removed and replaced with an implant, (b) an empty tooth socket from which a tooth has been removed, and (c) a portion of a previously placed dental implant;
positioning a second electrode on the mouthpiece at a second location of gingival tissue of the human, the second location of gingival tissue at least partially surrounding at least one of (a) the tooth to be removed and replaced with an implant, (b) the empty tooth socket from which the tooth has been removed, and (c) the portion of the previously placed dental implant; and
delivering an electrical current between the first electrode and the second electrode.

25. The method of claim 24, wherein the first electrode comprises one of an anodic electrode and a cathodic electrode, and
wherein the second electrode comprises the other of the anodic electrode and the cathodic electrode.

26. The method of claim 25, wherein the first location of gingival tissue comprises one of exterior gingival tissue and interior gingival tissue, and
wherein the second location of gingival tissue comprises the other of the exterior gingival tissue and the interior gingival tissue.

27. The method of claim 26, wherein the exterior gingival tissue is selected from the group consisting of buccal gingival tissue, facial gingival tissue, and vestibular gingival tissue.

28. The method of claim 26, wherein the interior gingival tissue is selected from the group consisting of lingual gingival tissue and palatal gingival tissue.

29. The method of claim 24, wherein the first location of gingival tissue comprises one from a group consisting of exterior gingival tissue and interior gingival tissue, and
wherein the second location of gingival tissue comprises the other from the group consisting of exterior gingival tissue and interior gingival tissue.

30. The method of claim 29, wherein the exterior gingival tissue is selected from the group consisting of buccal gingival tissue and vestibular gingival tissue, and
wherein the interior gingival tissue is selected from the group consisting of lingual gingival tissue and palatal gingival tissue.

31. The method of claim 24, wherein the first location of gingival tissue comprises one from a group consisting of buccal gingival tissue and lingual gingival tissue, and
wherein the second location of gingival tissue comprises the other from the group consisting of buccal gingival tissue and lingual gingival tissue.

32. The method of claim 24, wherein the tooth has been intentionally removed from the empty tooth socket.

33. The method of claim 24, wherein the tooth has been accidentally removed from the empty tooth socket.

34. The method of claim 24, further comprising the step of preventing conduction of electrical current from a third electrode to any other oral tissue of the human, the third electrode being electrically isolated from the first electrode and the second electrode.

35. The method of claim 34, wherein the preventing conduction step comprises refraining from placing the third electrode in an oral cavity of the human.

36. The method of claim 34, wherein the preventing conduction step comprises the step of deactivating the third electrode.

37. The method of claim 24, wherein the delivering step comprises the steps of:
electrically coupling the first electrode and the second electrode to a power supply; and
causing the power supply to discharge electrical current to one of the first electrode and second electrode.
